(19) United States
(12) Patent Application Publication
(10) Pub. No.: US 2009/0076516 A1
(43) Pub. Date: Mar. 19, 2009

(54) DEVICE AND METHOD FOR TISSUE RETRACTION IN SPINAL SURGERY

(76) Inventors: David Lowry, Holland, MI (US); Desmond O'Farrell, Grand Rapids, MI (US); Scott Tuinstra, Holland, MI (US); Roger Veldman, Hudsonville, MI (US)

Correspondence Address: SHAY GLENN LLP, 2755 CAMPUS DRIVE, SUITE 210, SAN MATEO, CA, 94403, US

(21) Appl. No.: 12/210,109

(22) Filed: Sep. 12, 2008

Related U.S. Application Data
(60) Provisional application No. 60/972,199, filed on Sep. 13, 2007.

(51) Int. Cl. A61B 17/58

(52) U.S. Cl. 606/90

(57) ABSTRACT

The invention relates to a system and methods for retracting soft tissue during spinal repair or reconstruction procedures, particularly procedures within intervertebral sites of degenerated discs and associated neural compression. A retractor system includes an implantable bone plate, two or more retractor blades, the bone plate and retractor blades being mutually engageable; and a mechanism to adjust the retractor blade relative to the bone plate. In some embodiments, the retractor system may be applied to span more than one intervertebral space. An implanted retractor system provides an aperture for a clear operating field, and protects collateral tissue. A method for retracting soft tissue to facilitate spinal surgery includes securing a bone plate to adjacent vertebral bodies, engaging one or more retractor blades to the bone plate; and adjusting the angular position of the retractor blade relative to the bone plate so as to retract tissue lying external to the bone plate.

### CROSS REFERENCE TO RELATED APPLICATIONS

This application claims priority to U.S. Provisional Patent Application No. 60/972,199 of Lowry et al., entitled “Device and method for tissue retraction in spinal surgery”, as filed on Sep. 13, 2007.

### FIELD OF THE INVENTION

The present invention relates to temporarily-inserted surgical devices that engage a surgically implanted device already in place. More particularly, such a temporarily inserted device may engage an implanted vertebral stabilizing bone plate for purposes of maintaining soft tissue retraction during spinal surgery to facilitate the surgery and to protect the tissue from accidental injury.

### INCORPORATION BY REFERENCE

All publications, patents and patent applications mentioned in this specification are herein incorporated by reference to the same extent as if each individual publication, patent or patent application was specifically and individually indicated to be incorporated by reference.

In particular, U.S. patent application Ser. No. 11/855,124 of Lowry et al. (filed on Sep. 13, 2007, entitled “Implantable bone plate system and related method for spinal repair”), U.S. Provisional Patent Application No. 60/972,192 of Lowry et al. (filed on Sep. 13, 2007, entitled “Transcorporeal spinal decompression and repair system and related method”) as well as the U.S. patent application (Atty. Docket 10323.704.200) of the same inventors and title being filed concurrently with the present application, and U.S. Provisional Patent Application No. 60/976,331 of Lowry et al. (filed on Sep. 28, 2007, entitled “Vertebrally mounted tissue retractor and method for use in spinal surgery”), and U.S. Provisional Patent Application No. 60/990,587 of Lowry et al. (filed on Nov. 27, 2007, and entitled “Methods and systems for repairing an intervertebral disk using a transcorporal approach”) are all incorporated by this reference.

### BACKGROUND OF THE INVENTION

The surgical removal of spinal disc or vertebral bone material and of adjacent tissue is commonly performed to address various disease states; typically the procedure is done to achieve a decompression of one or more neural elements and/or to stabilize the spine. One current surgical practice is to remove disc material from between adjacent vertebrae and any collateral tissue that impedes or complicates surgical access to the disc, achieve a neural decompression by excising the compressing pathology. Next an implant of bone or bone substitute material is inserted that induces bony growth therethrough, ultimately forming a solid fusion piece across the disc space. A bone plate is then typically applied to the anterior aspect of the spine and permanently fastened to two or more adjacent vertebrae across the implanted device within the disc space. The plate serves to secure the implant and to provide structural support and to benefit the biological fusion process by preventing relative motion between the vertebral segments and the fusion implant device. In an alternate procedure an artificial disc implant is inserted between the adjacent vertebrae so as to preserve motion across the disc space.

Retraction of surrounding soft tissues during these surgical procedures is highly beneficial toward the ends of preventing tissue intrusion into the exposed surgical field and providing protection against accidental injury to surrounding tissue from surgical instruments. Currently available retractor systems are prone to undesirable shifting or migrating within the wound. These systems typically rest spinal or nearby tissue, and are held in place only with a force that is sufficient to counteract impinging forces exerted by adjacent tissue. Other retractor systems are incorporated into a distractor mechanism, and anchor to the vertebral body by bone screws. Consequently, currently available retractor systems can be considered less than completely satisfactory for several reasons. For example, the field of view and working area within the surgical wound can be restricted by encroaching retractor devices and soft tissue, and they require frequent adjustment and repositioning during the surgery, significantly elevating the risk of injury to adjacent soft tissue structures with each adjustment and unnecessarily prolonging the procedure.

The current art includes several methods and devices used to maintain soft tissue retraction during spinal surgery. U.S. Pat. No. 5,795,291 of Koros discloses a soft tissue retractor system that uses a combination of blades that are inserted into soft tissue overlaying one or more vertebral bodies and then laterally spread by adjusting a top-mounted ratcheting frame to which the blades are rigidly mounted. U.S. Patent App. No. 2006/0084844A1 of Nehls discloses a soft tissue retractor using a combination of blades that has incorporated into the design a distractor device for achieving simultaneous soft tissue retraction and intervertebral distraction. U.S. Patent. App. No. 2003/0149341A1 of Clifton discloses a spinal retractor system that utilizes a combination of blades and anchors for simultaneous soft tissue retraction and intervertebral distraction.

### SUMMARY OF THE INVENTION

Conventional techniques in spinal fusion surgery typically make use of a stabilizing bone plate that is applied after tissue removal and after the insertion of a repair device. Since stabilizing bone plates are applied toward the end of the procedure, currently available soft tissue retractor systems cannot interact with these bone plates. However, as disclosed in U.S. patent application Ser. No. 11/855,124 of Lowry (entitled “Implantable bone plate system and related method for spinal repair”, filed on Sep. 13, 2007, and incorporated herein by this reference), a permanently implantable bone plate may be placed at a surgical site prior to substantial removal of tissue and prior to the insertion of a repair device. The invention now summarized here is directed toward a surgical device that includes a bone plate that is implanted before surgery, and which supports retractor blades for the purpose of maintaining soft tissue retraction during surgery. Embodiments of the inventive tissue retraction system require less manipulation during surgery than do currently available spinal retractor systems, thus placing collateral soft tissue at a lesser risk of damage, and generally improving the efficiency and safety of the surgical procedure.

The invention provides a retractor system for facilitating spinal surgery and methods of surgery that use the system. The retractor system includes an implantable bone plate configured to attach to at least one vertebral body (the plate having at least two laterally-spaced apart retractor blade engagement features), and at least two retractor blades, each blade having an upper aspect and a lower aspect, and a bone plate engagement feature on the lower aspect.

Some embodiments of retractor system include more than two retractor blades. In some embodiments, the bone plate is configured to span more than one intervertebral space. The bone plate and the retractor blades of the system may be particularly sized and configured for surgical sites in the cervical spine.

The retractor blade engagement feature of the bone plate and the bone plate engagement feature of the retractor blade are complementary and configured to provide a pivotable engagement of the blades to the bone plate, the pivotable engagement configured to allow variation of the angle included between the two blades when the blades are engaged to the plate. Some embodiments of the bone plate include an operating aperture that represents an open operating field when the system is implanted, and when the retractor blades have been opened to an operating angle.

Embodiments of the retractor blade have an upper aspect with a feature that is adapted to pivotably engage a complementary feature of an adjustment mechanism. The adjustment system, as included in some embodiments of the system, is adapted to adjust the angle included between the two blades when the bone plate and the retractor blades are engaged; such adjustment of the system, when the bone plate is implanted, can operate to retract soft tissue from around the bone plate. The adjustment mechanism may include a feature adapted to pivotably engage a complementary feature on the upper aspect of a retractor blade, as recited above. The adjustment mechanism may further include a setting mechanism that operates to hold the blades at an angle to which they have been adjusted.

In some embodiments of the system, the bone plate engagement feature of the retractor blade and the retractor blade engagement feature of the bone plate are mutually configured to form a passive engagement. This passive engagement is supported and held in place by the laterally-impinging force of retracted soft tissue when the system is implanted. In some embodiments of the system, the bone plate engagement feature of the retractor blade includes one or more protrusions adapted to engage the implantable bone plate.

In some embodiments of the system, the bone plate engagement feature of the retractor blade is adapted to serve as a fulcrum of pivotable rotation for the blade with respect to the bone plate. Further, in some embodiments, the retractor engagement feature of the bone plate is substantially outward-facing and the bone plate engagement feature of the retractor blade is substantially-inward facing such that the retractor blade is external to the bone plate when the bone plate and the retractor blade are engaged. In other embodiments, the engagement features of the bone plate and the retractor blades may be modified such that the retractor blades engage the bone plate in an articulating manner at a point internal to the periphery of the bone plate, or on the upper surface of the bone plate.

In some embodiments of the system, the bone plate includes two axially-oriented side rails and at least one connecting end bar, and the side rails and the at least one end bar define the operating aperture recited above. In some embodiments of the system, the bone plate includes two axially-oriented end bars and two parallel side rails, and the retractor blade engagement feature of the bone plate includes a depression in any of the end bars or the side rails.

In some embodiments of the system, the bone plate includes two side rails, and a first retractor blade is engageable with one of the side rails while a second retractor blade is engageable with the other side rail. In some embodiments of the system, the bone plate includes two end bars, the end bars including bone plate engagement features, and the retractor blade is engageable with at least one of the end bars.

While embodiments of the retractor system typically have retractor blades with pivotable engagements both at their upper aspect, with an adjustment mechanism, and at their lower aspect, with the bone plate, embodiments include options whereby such engagements may be pivotable but lockable, or whereby such engagements are rigid. For example, a retractor system may include an implantable bone plate (as summarized above), at least two retractor blades (as summarized), wherein the retractor blade engagement feature of the bone plate and the bone plate engagement feature of the retractor blades are complementary, and at least one of the two complementary engagements between the bone plate and the retractor is configured to be a pivotable engagement of the blade to the bone plate, the pivotable engagement configured to allow variation of the angle included between the two blades when the blades are engaged to the plate. In this example, therefore, one of the engagements between the blade and the bone plate can be rigid.

The invention, as recited above, also provides a method for spinal surgery that makes use of the above-summarized system. The method includes securing a bone plate to at least one vertebral body (the bone plate having at least two laterally spaced-apart retractor blade engagement sites), pivotably engaging a lower aspect of a retractor blade to each of the two engagement sites, and adjusting the angle included between the two blades so as to retract soft tissue adjacent to the bone plate. The method for spinal surgery, prior to implanting and securing the bone plate to a vertebral body, is typically preceded by exposing one or more vertebral bodies in a spinal column by anterior incision.

In embodiments of the method, the step by which the bone plate is secured to a vertebral body may include securing the bone plate to at least one vertebral body in the cervical spine, it may include securing the bone plate to adjacent vertebral bodies, and/or it may include securing the bone plate to a plurality of vertebral bodies.

In some embodiments of the method, the adjusting step includes pivotably-engaging an upper aspect of each blade to an adjustment mechanism. In some embodiments, the method further includes holding the blades at the angle to which the blades have been adjusted by the adjustment mechanism. In some embodiments of the method, the adjusting step provides a clear operating field for a surgical procedure, which is stabilized by setting the adjusted angle. In embodiments of the system where the bone plate has an operating aperture, the adjusting step provides a clear operating field above the aperture for a surgical procedure.

Following the adjusting step whereby soft tissue has been retracted, the method may further include performing a surgical procedure in a clear operating field provided by the soft tissue having been retracted. And finally, the method may include leaving the bone plate attached to the vertebral body after the surgical procedure has been performed.

### BRIEF DESCRIPTION OF THE FIGURES

FIG. 1 is a side elevation view of the bone plate with receiving means for soft tissue retractors.

FIG. 2 is a side elevation view of a retractor blade.

FIG. 3 is a front elevation view of a retractor blade.

FIG. 4 is a side view of retractor blades prior to engagement of bone plate.

FIG. 5 is a side view of retractor blades engaged to bone plate.

FIG. 6 is an exploded perspective view of a retractor blade and bone plate.

FIG. 7 is a top plan view of an implanted bone plate with two lateral wall retractors.

FIG. 8 shows a perspective view of an implanted bone plate.

FIG. 9 shows a perspective view of the implanted bone plate as in FIG. 8, with two retractor blades now pivotably engaging the bone plate at their lower aspect and pivotably engaging an adjustment mechanism at their upper aspect; the blades are shown in an upwardly convergent configuration, as they would be prior to retracting adjacent soft tissue.

FIG. 10 shows a perspective view of the implanted bone plate as in FIG. 9, but with two retractor blades now adjusted to an upwardly open configuration, as they would be when retracting adjacent soft tissue.

### DETAILED DESCRIPTION OF THE INVENTION

The inventive system provided herein for use in surgical spinal repair or reconstruction procedures may include (1) an implantable bone plate or frame with retractor engagement features, such as depressions within the two lateral faces of the device that are adapted to receive and dock with complementary bone engagement features on the retractor blades such may be located on the tips of right and left retractor blades; (2) right and left lateral retractor blades with bone plate pivotable engagement features, such as one or more protrusions on the distal tips that are configured to pivot on the lateral faces of the bone plate; (3) top and bottom (cranial/caudal) retractor blades, similarly attachable and pivotable with respect to the implantable bone plate; (4) a ratcheting or adjusting mechanism by which the blades may be adjustably and controllably separated to a desired distance from each other or distanced with respect to a midline over the bone plate; and (5) a pivoting or rotating element engagement to the ratcheting mechanism whereby the right and left blades can pivot with respect to the ratcheting mechanism.

Retractor blade embodiments may be made of stainless steel, but other metallic and radiolucent materials or non-metallic materials may be used as well. The blade may be curved with rounded edges to minimize the risk of a laceration of adjacent retracted soft tissue. The long axis of the left and right blades may be straight or, alternatively, offset with respect to the lateral edge of the implanted plate so that the tissue retracted resides closer or further from the midline of the body than the lateral edge of the plate itself. The blade angle along the long axis with respect to the plate may also vary in the cranial-caudal dimension to optimize the surgeon's sight line.

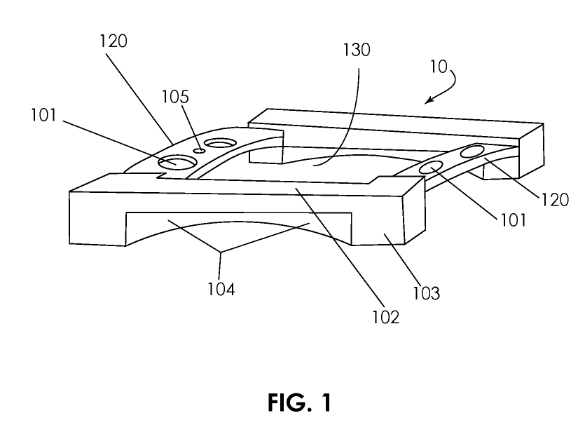

The interface between the retractor blade and the ratcheting or adjustment mechanism may be adjustable, permitting the relationship of the blade with respect to the ratcheting mechanism to be fixed relative to one another, or alternatively, adjustable so as to allow the retractor blades to pivot within the confines of the attachment. In an embodiment described in more detail below and as seen in FIG. 1, the tips of the retractor blades interact with small depressions on the lateral side wall of the bone plate and are held in place by compression from adjacent soft tissue.

In one embodiment, the retractor blades have one or more additional protrusions 112 on the distal tips and on the surface facing an anterior cervical spinal plate that relate to a corresponding depression or groove in the surface of the plate; the corresponding relationship and configuration being adapted to assist in the manipulation of the plate within a surgical exposure. This configuration permits the manipulation of the plate into the final position desired for implantation with the use of one retractor blade only.

In some embodiments, multiple retractor blades may engage a bone plate that spans more than one vertebral segment. In other embodiments, additional retractor blades that also similarly relate to the implanted plate may be used to provide cranial-caudal wall soft tissue retraction.

The inventive system and its component devices can be applied in a method of spinal surgery, or more particularly to a method of providing surgical access and a clear operating space so spinal surgery can proceed optimally. In another aspect, embodiments of the invention provide a method of retracting soft tissue from an intervertebral surgical site, such surgery typically being that of vertebral body fusion. Briefly, some embodiments of the method include securing a bone plate to adjacent vertebral bodies (the bone plate having an operating aperture that frames an operating space within the plate and one or more retractor blade engagement sites on external surfaces of the plate), engaging one or more retractor blades to the one or more retractor blade engagement sites of the bone plate; and retracting tissue lying external to the bone plate by spreading at least the upper portion of the blades and widening their included angle.

Various embodiments and features of the invention as described above are depicted as examples in FIGS. 1-10 and will now be detailed.

FIG. 1 is a side elevation view of an embodiment of a permanently implantable bone plate 10. The bone plate 10 is of a generally rectangular form that includes two substantially parallel end bars 120 and two substantially parallel side rails 102 that collectively frame a central surgical working space (aperture or window) 130. When the device is implanted in situ, the side rails 102 may be referred to being either a left or a right side rail, depending on their position. When the device is implanted in situ, the end bars 120 may be referred to being either caudal or cranial, depending on their relative position.

End bars 120 include receiving holes 101 of various sizes and shapes as appropriate for the placement of bone screws or other fastening elements. Additional smaller receiving holes 105 permit screws to secure a retention plate or member for purposes of preventing intervertebral implant and/or bone screw migration. The corners 103 of the bone plate 10 are adapted to serve as contact pads that rest on the spine when the device is implanted, and permit an optimal plate contact with an uneven bone surface. Beneath the side rails 102, along the lateral wall of the bone plate 10, are depressions 104 that are adapted to accommodate or engage complementary features on a surface of soft tissue retractor blades.

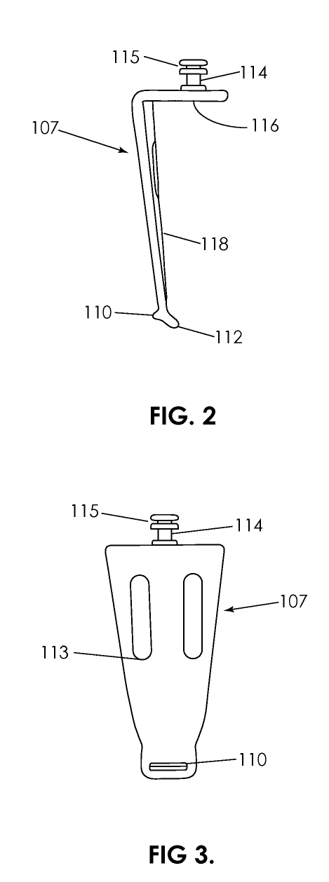

FIG. 2 is a side elevation view of a retractor blade 107 that includes a side plate 118 and a head plate 116 that are configured at approximately a right angle with respect to each other. Typically, the angle is more acute than a right angle, in the range of about 80 to 89 degrees, for example. In some embodiments, however, the angle between the side plate and the head plate may be 90 degrees or more. The side plate 118 includes a bone plate engagement feature 110 in the form of a small outward protrusion, for example, near the tip of the blade that is adapted to engage the implantable bone plate 10, as shown in FIG. 1. A curved distal tip 112 of retractor blade 107 is adapted to facilitate tissue control during surgical exposure and soft tissue retraction.

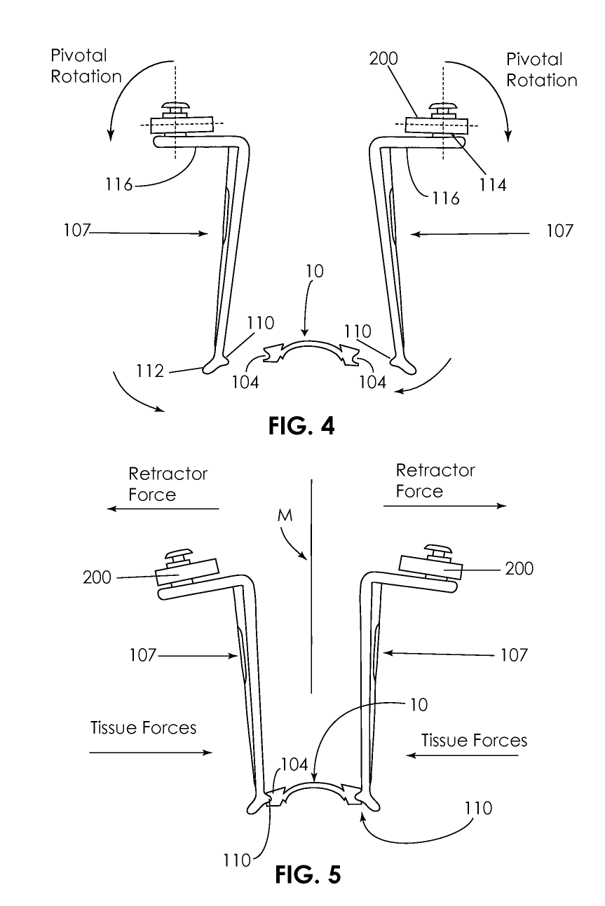

A centrally-mounted post 114 on the head plate 116 is adapted to facilitate continuously variable interaction between the retractor blade 107 and a ratcheting or blade angle adjustment mechanism 200, as partially depicted in FIGS. 4 and 5, which holds the blade in place. An extension element 115 on the centrally mounted post 114 permits interaction between a retractor blade handle and the retractor blade 107. FIG. 3 is a front elevation view of a retractor blade 107 that shows an exemplary bone plate engagement feature, a small outward protrusion 110. Shown also are visual access features in the form of slots 113 on the blade wall that are adapted to permit visibility of tissue during a surgical procedure.

FIG. 4 is a side view of two retractor blades 107 situated on either side of a bone plate 10 prior to the engagement of the two blades with the plate as occurs when the device is being used during a surgical procedure. The retractor blades 107 may be temporarily positioned to rigidly maintain soft tissue retraction during surgical exposure and for the delivery/attachment of the bone plate 10. In one embodiment, pivotal rotation (indicated by arrows) occurs in one or both arms of a ratcheting spreader device that is adapted to permit the tip of the retractor blades 107 to rotate inward and engage that bone plate 10. In an alternate embodiment, relative pivotal rotation may occur at the retractor blade-ratcheting retractor arm interface.

FIG. 5 is a side view of two retractor blades 107 and a connecting bone plate 10 that shows the common site of engagement 116 of the protruding feature 110 of retractor blades to the depressed space 104 beneath the side rails 102 of the bone plate 10, after such engagement has been made. The ratcheting retractor arms 200, as partially depicted, are adapted to exert a force away from a midline M between the substantially parallel surfaces of the two respective blades 107, further separating the distance between the two retractor blades. Because the retractor blade 107 and ratcheting retractor arm 200 engagement permits pivotal rotation, the distal tips of the retractor blade remain positively engaged in the bone plate 10 by medially-directed forces from adjacent soft tissue structures (per the arrows), and the retractor blades pivot about their site of engagement at the bone plate, with the distal tips of the retractor blades acting as a fulcrum for the blade as a whole.

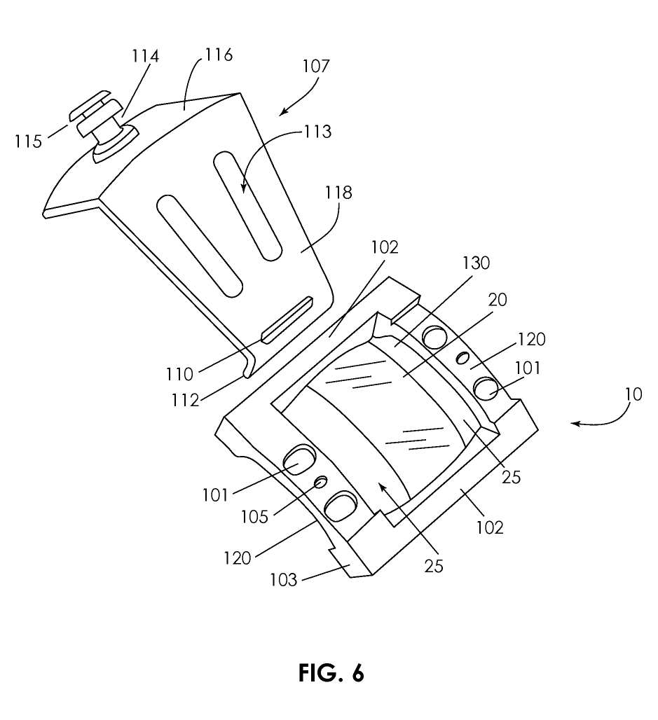

FIG. 6 is an exploded top perspective view of the bone plate 10 and one of the two retractor blades 107, the view showing how the retractor blade aligns for eventual interaction with plate 10. A small outward protrusion 110 near the tip of the retractor blade 107 inserts beneath the side rail 102 along the lateral wall of the plate 10. The tip of the retractor blade has a rounded curve 112 adapted to optimize control of soft tissue during retraction by providing a fulcrum with a continuous surface contact. Vertebral disc tissue 20 can be seen between two adjacent vertebral bodies 25 through the central void of central working space 130, as it would appear from the perspective of a surgeon, when the device is placed in situ.

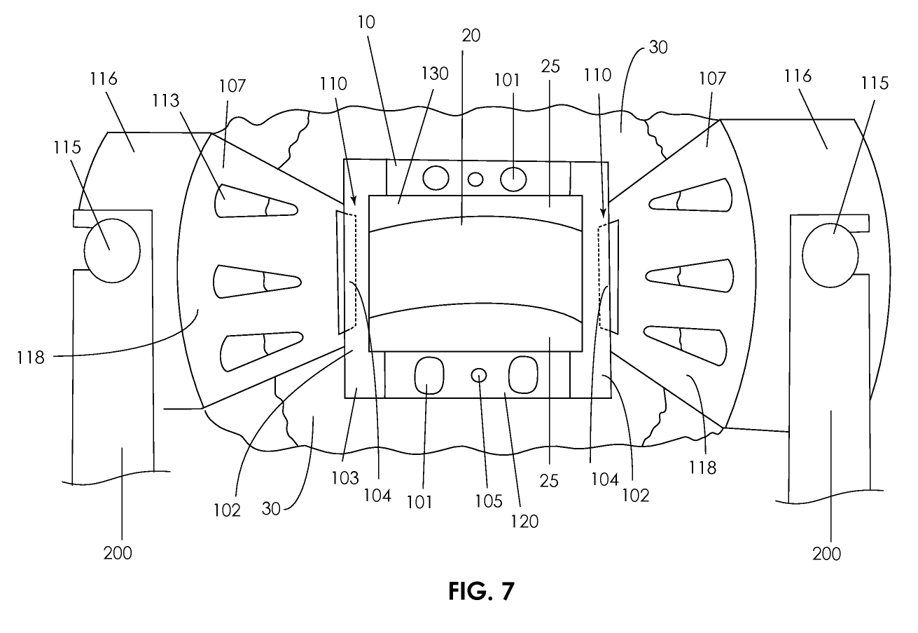

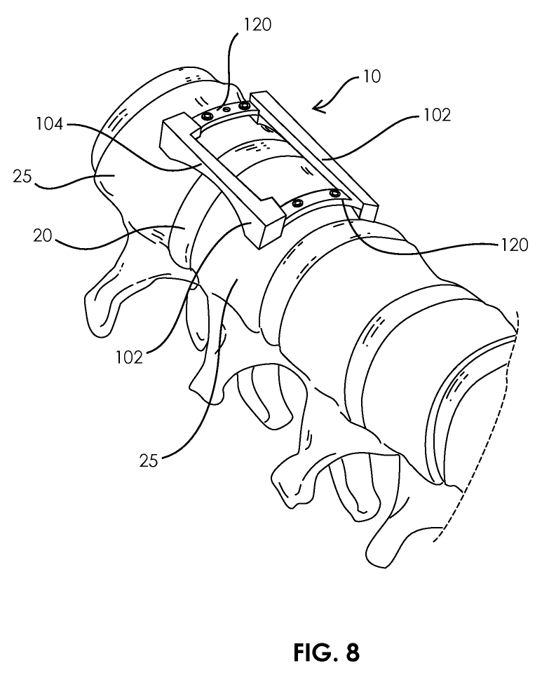

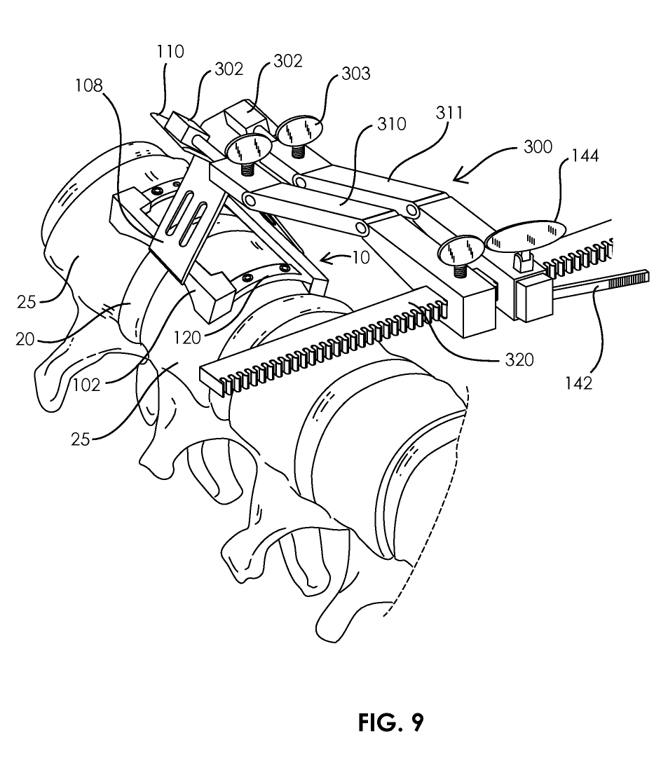

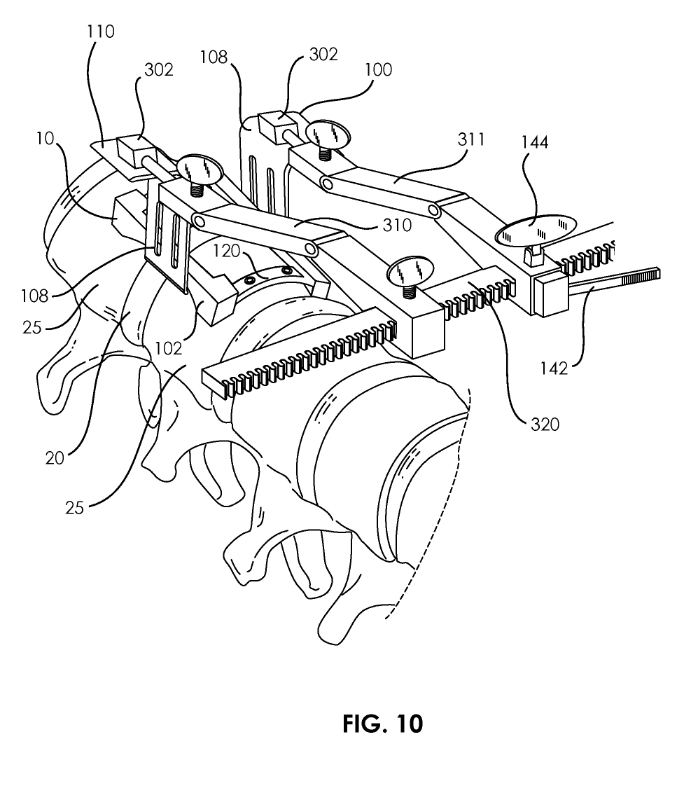

FIG. 7 is a top plan view of two retractor blades 107 outwardly spread and a bone plate 10 there between. The view shows the interaction of protrusions 110 of the retractor blades with side wall depressions on the bone plate 10 in situ, surrounded by retracted soft tissue 30. The impinging force of the soft tissue stabilizes the engagement between the retractor blade and the bone plate. In some embodiments, the respective positions of the protrusions 110 on the retractor blades 107 and the depressions 104 on plate 10 may be reversed. Alternatively, other inter-engaging features may be employed instead of the exemplary protrusions and depressions to allow retractor blades to temporarily and pivotably engage with plate. For example, a curved tongue may be used instead of protrusion 110, and a mating slot through plate 10 may be used instead of depression 104. The upper aspect or headplate 116 of retractor blades 107 can be seen engaging an angular adjustment mechanism 200. (Details of the angular adjustment mechanism are as embodiment 300 in FIGS. 9 and 10). The sideplates 118 of the retractor blades 107 can be seen holding retracted soft tissue 30 back from the region over the implanted bone plate 10, thereby creating an operating field over the aperture 130 in the bone plate.

FIGS. 8-10 provide a series of views of an implanted retractor system, including a bone plate 10, retractor blades 108, and an adjustment mechanism 300, and depict aspects of the method by which they are used.

FIG. 8 shows a perspective view of an implanted bone plate 10. FIG. 9 shows a perspective view of the implanted bone plate 10 as in FIG. 8, with two retractor blades 108 now pivotably engaging the bone plate at their lower aspect 109 and pivotably engaging an adjustment mechanism 300 at their upper aspect 110. The bone plate is implanted on vertebral bodies 25 overlaying intervertebral disc 20. The blades are shown in an upwardly convergent configuration, as they would be prior to retracting adjacent soft tissue (not shown). The upper aspect 110 of the retractor blades 108 shown here differ compared to the upper portion of retractor blades 107 as shown in FIGS. 2-6, in that the embodiment 108 does not have an extension element 115 on the centrally-mounted post 114. The broad angled upper aspect 110 of retractor blade 108 is engageable with a pivotable engagement feature 302 of adjustment mechanism 300. This form of pivotable engagement between a retractor blade and an adjustment mechanism is merely exemplary and is not intended to be limiting. A number of embodiments of mutually engageable features of a retractor blade and an adjustment mechanism will be apparent to those skilled in the art; any such configuration or feature as it applies to either the retractor blade or the adjustment mechanism such that the engagement of the retractor blade and the adjustment mechanism as a whole is pivotable is included as an embodiment of the invention.

Further shown in FIG. 9 (and FIG. 10) are rotation locking elements 303 of the adjustment mechanism 300 which are operable to prevent pivoting of the engagement between a retractor blade 108 and the adjustment mechanism. The depicted rotation locking element is merely exemplary and is not intended to be limiting. A number of embodiments of rotation locking element will be apparent to those skilled in the art and their form will depend on the precise of the pivotability of the engagement between the retractor blade and the adjustment mechanism; any such configuration or feature as it applies to either the retractor blade or the adjustment mechanism such that pivotability of the engagement of the retractor blade and the adjustment mechanism as a whole is reversibly lockable is included as an embodiment of the invention.

It will also be apparent that some of the features of the adjustment mechanism 300 resemble those of the retractor frame of Koros (U.S. Pat. No. 5,795,291), as previously referenced and incorporated. For example, the arms 310 and 311, the crank handle 144, the quick release lever 142, and the toothed cross brace 320 of adjustment mechanism 300 are all broadly similar to the device of Koros. Further, the broad mechanism by which the adjustment mechanism adjusts the angle or distance between retractor blades 108 is also similar to that of Koros. A difference between the adjustment device 300 and the device of Koros relates to the nature of the engagement between the adjustment device and the retractor blades. Whereas the engagement between the frame of Koros and the retractor blades is rigid, and disallowing of pivotable movement of the retractor blades, the engagement between retractor blades 108 and the adjustment mechanism 300 is pivotable. The pivotable engagement, however, is also lockable through the intervention of rotation locking elements 303. In various embodiments of the method of using the retractor system, there may be benefit and occasion to lock and unlock rotation elements.

Although not specifically related to the structural difference between the adjustment mechanism 300 and the device of Koros, the functional result of spreading and closing the arms 310 and 311 of adjustment mechanism 300 differs from the result of spreading and closing the arms of the Koros device by virtue of the pivotability also of the engagement of the retractor blades 108 and the bone plate 10. The retractor blades of Koros are rigidly attached at a right angle or an approximate right angle with respect to the frame, and the blades are not engageable to an implanted bone plate. Accordingly, the blades maintain such approximate right angle orientation regardless of the width by which the adjustment frame arms are spread. In the present system, the spreading and closing of arms 310 and 311 varies the included angle between the arms as well as the distance separating the upper aspects 110 of the retractor blades.

FIGS. 9 and 10, for example, show a difference in angles between the blades according to the varying distance between arms 310 and 311, as provided by the adjustment mechanism 300. In FIG. 9 the retractor blades 108 are tilted inwardly toward each other, meeting at a medial center over the bone plate 10. In FIG. 10, the arms 310 and 311 have been spread, by the action of the crank handle 144, and accordingly, the retractor blades 108 are approximately parallel to each other. As the arms 310 and 311 are moved farther apart, it can be understood that the retractor blades begin to form an ever increasingly open, upward-facing angle.

As described above and depicted in FIGS. 4 and 5, as well as in FIGS. 9 and 10, the retractor blades are situated externally with respect to the bone frame 10. The lower aspect 109 of the retractor blade (see as distal tip 112 in FIG. 4) passively but securely engages an external aspect (seen as depression 104 in FIG. 1) of side rail 102. Unseen in FIGS. 9-10 is the soft tissue surrounding the implanted system, but it can be understood (and as indicated in FIGS. 4 and 5), such surrounding soft tissue is spread apart by widening the distance (widening the angle) between the retractor blades, and such spread apart tissue, being compressed, provides a countering inward pressure that maintains the engagement and stability of the lower aspect of the retractor blades against the bone plate. It can be understood, accordingly, that the lower aspect or distal tip of retractor blades acts as a fulcrum around which the blades can pivotable rotate, as provided by the varying distance between the upper aspects of the blades, as controlled by the adjustment mechanism 300. The distance or variable angle between the blades can further be set and stabilized by the intervention of a mechanism such as that provided by set screw 330.

FIG. 10 shows a perspective view of the implanted bone plate 10 as in FIG. 9, but with two retractor blades 108 now adjusted to an upwardly open configuration, as they would be when retracting adjacent soft tissue. It can be seen that the retractor blades in FIG. 10 are approximately parallel, and that this is approximately an intermediate position with regard to the range of angles that can be imposed on the blades by the adjustment mechanism. If the arms 310 and 311 were to be moved further apart, the included open angle between the blades could be advanced, for example to about 45 degrees or wider. The range of the opening angle is not practically limited by the adjustment mechanism itself, but more practically by the specifics of the anatomy of the surgical site and the judgment of the operating surgeon.

Also, as seen in FIGS. 9 and 10, as well as in FIGS. 6 and 7, by retracting soft tissue adjacent or external to the bone plate and retractor blades, a clear operating field is created over a site of interest, typically an intervertebral site. The operating field is generally bounded at its base by the aperture included within the bone plate, and proximal from the base, by the angularly expanded retractor blades.

Although the embodiments of the system depicted show a typical configuration whereby the retractor blades are external to the bone plate at their site of interaction, other configurations are included as embodiments of the invention. It can be understood, for example, that the retractor blades could interact or engage a bone plate at a site internal to the plate, or at some point within the periphery, or by interaction with a stop feature on the upper surface of the plate. The feature common to these alternative engagement sites is that laterally impinging force from retracted tissue supports and stabilizes the engagement, and freely allows pivoting of the retractor blade at that point.

Further, although embodiments of the system that have been described in some detail and depicted are those where the retractor blades are pivotably engaged both on their lower and upper aspects (pivoting at the engagement site on the bone plate at their lower aspect, and pivoting at the engagement site with the adjustment mechanism at their upper aspect), the system includes embodiments where such pivoting engagement can be lockably prevented from moving. The rotation locking elements 303 of the adjustment mechanism 300, as seen in FIGS. 9 and 10, for example, can be utilized to prevent pivoting at that site. With regard to the engagement of the retractor blade with the bone plate, the complementary engagement features of the retractor blade and the bone plate may include a rigid feature or features insertable into a receiving slot or hole, or plurality of such receiving sites, such that the engagement as a whole is rigid. Thus, by a combination of pivotable engagements, pivotable but lockable engagements, and rigid engagements, a number of embodiments with varying options related to angular stability and variability are included within the scope of the invention.

The described retractor system may be employed in various methods of performing spinal surgery, such as vertebral fusion procedures, but more generally any procedure that benefits from having a well exposed surgical site on vertebral bodies. Embodiments of the method include steps of securing a bone plate to adjacent vertebral bodies, the bone plate having one or more retractor blade engagement sites, engaging one or more retractor blades to the one or more retractor blade engagement sites of the bone plate, and adjusting the angular position of the retractor blade relative to the bone plate so as to retract tissue lying external to the bone plate. By such retraction, a surgical field is established that is visually clear for an operating surgeon, and which further provides physical access for surgical instruments, or for passage of surgical implants, or tools that deliver surgical implants.

While the exemplary embodiments of the invention described herein relate to the performance of surgical repair procedures in the cervical spine, it may be understood that adaptations of the system can be utilized at other skeletal sites and in other orthopedic procedures where a bone plate is mounted permanently or temporarily on bone tissue.

What is claimed is:

1. A retractor system for facilitating spinal surgery in an intervertebral space, comprising:
an implantable bone plate configured to attach to at least one vertebral body, the plate including at least two laterally-spaced apart retractor blade engagement features; and
at least two retractor blades, each blade having an upper aspect and a lower aspect, and a bone plate engagement feature on the lower aspect,
wherein the retractor blade engagement feature of the bone plate and the bone plate engagement feature of the retractor blade are complementary and configured to provide a pivotable engagement of the blades to the bone plate, the pivotable engagement configured to allow variation of the angle included between the two blades when the blades are engaged to the plate.

2. The retractor system of claim 1 wherein the retractor system is sized and configured for facilitating surgery in the cervical spine.

3. The retractor system of claim 1 wherein the upper aspect of each retractor blade includes a feature adapted to pivotably engage a complementary feature of an adjustment mechanism.

4. The retractor system of claim 1 further comprising an adjustment mechanism adapted to adjust the angle included between the two blades when the bone plate and the retractor blades are engaged, wherein such adjustment of the system, when the bone plate is implanted, is operable to retract soft tissue from around the bone plate.

5. The retractor system of claim 4 wherein the adjustment mechanism includes a feature adapted to pivotably engage a complementary feature on the upper aspect of a retractor blade.

6. The retractor system of claim 5 further including a retractor blade that is adapted to pivotably engage the complementary engagement feature of the adjustment mechanism, wherein an engagement formed between the adjustment mechanism and the retractor blade is pivotable and lockable.

7. The retractor system of claim 4 wherein the adjustment mechanism includes a setting mechanism operable to hold the blades at an angle to which they have been adjusted.

8. The retractor system of claim 1 wherein the bone plate comprises at least one operating aperture.

9. The retractor system of claim 1 comprising more than two retractor blades.

10. The retractor system of claim 1 wherein the bone plate is configured to span more than one intervertebral space.

11. The retractor system of claim 1 wherein the bone plate engagement feature of the retractor blade includes one or more protrusions adapted to engage the implantable bone plate.

12. The retractor system of claim 1 wherein the bone plate engagement feature of the retractor blade and the retractor blade engagement feature of the bone plate are mutually configured to form a passive engagement, the engagement supported by laterally-impinging force of retracted soft tissue when the system is implanted.

13. The retractor system of claim 1 wherein the bone plate engagement feature of the retractor blade is adapted to serve as a fulcrum of pivotable rotation for the blade with respect to the bone plate.

14. The retractor system of claim 1 wherein the retractor engagement feature of the bone plate is substantially outward-facing and the bone plate engagement feature of the retractor blade is substantially-inward facing such that the retractor blade is external to the bone plate when the bone plate and the retractor blade are engaged.

15. The retractor system of claim 1 wherein the bone plate includes two parallel side rails and at least one connecting end bar, and wherein the side rails and the at least one end bar define an operating aperture.

16. The retractor system of claim 1 wherein the bone plate includes two parallel end bars and two parallel side rails, and wherein the retractor blade engagement feature of the bone plate includes a depression in any of the end bars or the side rails.

17. The retractor system of claim 1 wherein the bone plate comprises two parallel side rails, and wherein a first retractor blade is engageable with one of the side rails, and a second retractor blade is engageable with the other side rail.

18. The retractor system of claim 1 wherein the bone plate comprises two parallel end bars, the end bars including bone plate engagement features, and further wherein the retractor blade is engageable with at least one of the end bars.

19. A retractor system for facilitating spinal surgery in an intervertebral space, comprising:
an implantable bone plate configured to attach to at least one vertebral body, the plate including at least two laterally-spaced apart retractor blade engagement features; and
at least two retractor blades, each blade having an upper aspect and a lower aspect, and a bone plate engagement feature on the lower aspect,
wherein the retractor blade engagement feature of the bone plate and the bone plate engagement feature of the retractor blades are complementary, and at least one of the two complementary engagements between the bone plate and the retractor is configured to be a pivotable engagement of the blade to the bone plate, the pivotable engagement configured to allow variation of the angle included between the two blades when the blades are engaged to the plate.

20. A method for spinal surgery, comprising:
securing a bone plate to at least one vertebral body, the bone plate having at least two laterally spaced-apart retractor blade engagement sites; pivotably engaging a lower aspect of a retractor blade to each of the two engagement sites; and
adjusting the angle included between the two blades so as to retract soft tissue adjacent to the bone plate.

21. The method of claim 20 wherein the securing step includes securing the bone plate to at least one vertebral body in the cervical spine.

22. The method of claim 20 wherein the securing step includes securing the bone plate to adjacent vertebral bodies.

23. The method of claim 20 wherein the securing step includes securing the bone plate to a plurality of vertebral bodies.

24. The method of claim 20 wherein the adjusting step includes pivotably-engaging an upper aspect of each blade to an adjustment mechanism.

25. The method of claim 20 further including holding the blades at the angle to which the blades have been adjusted.

26. The method of claim 20 further comprising, prior to attaching the bone plate, exposing one or more vertebral bodies in a spinal column by anterior incision.

27. The method of claim 20 wherein in the adjusting step provides a clear operating field for a surgical procedure.

28. The method of claim 27 wherein in the bone plate has an operating aperture and wherein the adjusting step provides a clear operating field above the aperture for a surgical procedure.

29. The method of claim 20 further comprising performing a surgical procedure in a clear operating field provided by the soft tissue having been retracted.

30. The method of claim 29 further comprising leaving the bone plate attached to the vertebral body after performing the surgical procedure.
